(19) United States
(12) Patent Application Publication
(10) Pub. No.: US 2003/0068391 A1
(43) Pub. Date: Apr. 10, 2003

(54) Ingestible nerve and circulatory nutritional formulation

(76) Inventors: Dennis H. Harris, Scottsdale, AZ (US); Ronald E. General, Scottsdale, AZ (US); Robert Martin, Scottsdale, AZ (US)

Correspondence Address: JOSEPH W MOTT, JENNINGS STROUSS & SALMON PLC, 201 EAST WASHINGTON STREET, 11TH FLOOR, PHOENIX, AZ, 85004-2385, US

(21) Appl. No.: 10/264,589

(22) Filed: Oct. 3, 2002

Related U.S. Application Data
(60) Provisional application No. 60/326,784, filed on Oct. 4, 2001.

(51) Int. Cl. A61K 35/78; A61K 31/375; A61K 31/385; A61K 31/355; A61K 33/32; A01N 31/04; A61K 31/07

(52) U.S. Cl. 424/750; 424/769; 424/643; 514/440; 514/458; 514/474; 514/725

(57) ABSTRACT

An ingestible nerve and circulatory nutritional formulation is disclosed, comprising an antioxidant portion, an anti-inflammatory portion, a circulatory enhancement portion, a vasodilator portion, a nerve growth, conduction and regeneration portion, a glycemic control portion, a sorbitol inhibitor portion a lipid reduction portion, a mitochondrial activation portion and a pancreatic stem cell support element portion.

This application is a continuation-in-part of provisional patent application No. 60/326,784, filed Oct. 4, 2001.

### BACKGROUND

According to Robert E. Schmidt, M.D., Ph.D., professor of pathology at Washington University School of Medicine, as many as 60 percent of people with diabetes have some damage to the peripheral nervous system. And, according to the American Diabetes Association, almost 20,000,000 cases of diabetes have been diagnosed in the United States of America. The prevalence of peripheral neuropathy increases with the duration of diabetes, so that 25 years after the initial diagnosis of diabetes, the prevalence is 50 percent or greater. Although diabetes is the leading cause of peripheral neuropathy, there are hundreds of different causes of neuropathy.

There are many complications of neuropathy, including, but not limited to the following: Impotence, constipation, diarrhea, urinary incontinence, cardiovascular complications, muscle weakness, orthostatic hypotension, dizziness, pain, numbness, crawling and/or prickling sensations, tingling sensations, pins-and-needles sensations, burning sensations, and hypersensitivity of nerves

Neuropathy is the wasting and inflammation of nerve tissues. Peripheral neuropathy is damage to nerves that connect peripheral (outlying) portions of the body (especially the feet, toes, legs, arms, and hands) to the brain and spinal cord. It may involve only one nerve or several nerves. Diseases that cause peripheral neuropathies may either be acquired or inherited; in some cases, it is difficult to make that distinction.

The diabetes-peripheral neuropathy link has been well established. Hyperglycemia, which has emerged as a major risk factor for the development of diabetic neuropathy, may affect the peripheral sensory nerves through a variety of mechanisms:

a.) Intracellular sorbitol accumulation.

b.) Decreased neuronal blood flow, indirectly leading to peripheral nerve hypoxia.

c.) Auto-oxidation of glucose causing increased production of reactive oxygen species.

d.) Formation of advanced glycation end products (AGEs) by nonenzymatic glycation of proteins.

e.) Reduced synthesis of vasoactive prostanoid in the vasa nervorum leading to reduced endoneural blood flow and nerve hypoxia.

f) Apoptosis associated with mitochondrial dysfunction.

Loss of protective sensation from nerves gives rise to unperceived traumas and pressures during daily ambulation, and the loss of extremity spacial awareness and coordination increases the likelihood of injuries to soft and hard tissue structures of the body. Drops in blood pressure when standing may cause dizziness or dangerous falls. This can be especially problematic to diabetic patients, due to an already existing slow healing response. Additionally, hypersensitivity may cause pain, agitation, anxiety, and loss of sleep. Further, if nerve, muscle, skin, bone, organs, and glandular tissues become compromised, immunologic challenges may also ensue.

A reduction of proper circulation secondary to becoming more sedentary gives rise to further overall physiologic shutdown including muscle shrinkage or atrophy. This can become a factor, due to movement and exercise avoidance. Once the overall aerobic capacity of the body begins to wane via lack of proper cardiopulmonary stimulus, many catastrophic medical events may eventually unfold.

Although many pharmacologic treatments for the symptomatic treatment of peripheral neuropathy exist, no specific formulation has attempted to address the underlying nutritional or metabolic factors. Therefore, it is the object of the present invention to provide a unique combination of vitamins, minerals, herbs, amino acids, and other elements that will stimulate the repair and growth of damaged nerve tissue, prevent nerve tissue dysfunction from occurring, restore blood vessel integrity, improve insulin production and reduce insulin resistance, support immunologic function, stimulate peripheral circulation, provide antioxidant nutrients to the nerves and blood vessels and help reduce lipid levels.

### SUMMARY OF THE INVENTION

This invention overcomes the deficiencies of other approaches by combining specific ingredients to accomplish particular targetted benefits to neural and circulatory systems.

Generally speaking, the invention consists of a combination of nutritional ingredients which, when used separately or together, may lead the improvement of nerve and/or circulatory function. This invention, referred to hereinafter as The Formula, contains the following potential(s): free radical scavenger/antioxidant potential, vasodilator/circulatory venous support potential, nerve growth/regenerative factors as well as nervous maintenance potential, insulin resistance mediators/precursors, sorbitol inhibition potential, microcirculation protection and anti-inflammatory action, mitochondrial cell rehabilitation and lipid reduction, nerve conduction velocity facilitation potential, glycemic control/potentiators, autonomic and hereditary motor and sensory nerve facilitation/enhancement, an anti-inflammatory potential, pancreatic stem cell/insulin producing complement.

Varying ranges of the different ingredients are contemplated depending upon the condition to be treated. Some specific embodiments will be utilized in the following detailed description of the invention for illustrative purposes. Furthermore, the ingredients may be varied within a wide range, or additional ingredients added, in order to adapt the Formulation for a specific delivery system, such as spray, lozenge, dermal, intranasal, intramuscular, and intravenous.

### DETAILED DESCRIPTION OF THE INVENTION

Many clinical experiments have proven that the primary elements leading to nerve and circulatory complications in diabetics are the inability to obtain enough essential nutrients via dietary means or secondary clinical or subclinical malabsorption syndrome. In order for nervous system cells to function in an orderly fashion and survive, they rely upon nutrients delivered via the various blood delivery networks. Once blood flow is compromised due to poor circulation, and nerve cell dependent nutrients diminished or not supplied at all, nervous system tissues will begin to atrophy and die. The notion of targeting multiple mechanisms simultaneously by administering a combination of bioavailable nutrients is therefore winning converts among clinical investigators. The present invention corrects these deficiencies in the following manner: 1) Reduce auto-oxidation of glucose causing a reduction of cell destroying reactive oxygen species/free radicals; 2) Reduce formation of advanced glycation end products (AGEs.) by nonenzymatic glycation of proteins, thereby improving circulation capacity to all tissues, including nerves; 3) Increase neuronal blood flow, indirectly leading to peripheral nerve oxygenation; 4) Reduce intracellular sorbitol accumulation; 5) Improve microcirculation at the level of the vasa nervorum; 6) Improve nerve cell growth and regeneration; 7) Provide, anti-inflammatory response; 8) Reduce the likelihood of future generations becoming diabetic; 9) Increase nerve transmission/conduction; 10) Improve glycemic control, and therefore halt the progression of diabetes and its complications, including neuropathy.

These characteristics, along with others available when the formula is modified, make this invention an effective treatment modality for many complications associated with dysglycemic, dysfunctional conditions, including but not limited to, varicose veins, peripheral vascular disease, phlebitis, intermittent claudication, vasculitis, spider veins, muscle wasting, nerve tissue atrophy, poor circulation, cold feet, burning feet, extremity hyperesthesia, hip fracture secondary to falls associated with orthostatic hypotension, hypesthesia, neurodynia, impotence, diarrhea, constipation, and sleeplessness due to nerve dysfunction.

The free radical/antioxidant potential is provided primarily by Vitamins C, A, E, alpha lipoic acid, and zinc as well as other ingredients in the Formula. It is well established that oxidative cell damage secondary to excessive reactive oxygen species free radical activity can be quenched and/or neutralized by antioxidant augmentation.

The vasodilator/circulatory support is provided by Vitamin B-3 (niacin), Vitamin B-1 (Thiamine) and Horse Chestnut extract (HCE) (aesculus hippocastanum) and contains aescin (a saponin). It is well established that Vitamin B-3 (niacin) possesses vasodilatory effects. Phytomedicines with a venoactive effect like (HCE) are commonly used in the U.S. and Europe to treat venous insufficiency. The vasoprotective action of niacin is also important in the treatment of venous stasis.

The nerve growth/regeneration and maintenance potential is provided by Vitamin B-12 (Methylcobalamin), and Vitamin B-1 (Thiamine). It is well established, that Vitamin B-12 prevents nerve damage and has been shown to regenerate nerves in human with peripheral neuropathies. One of the symptoms of Vitamin B-1 deficiency is numbness of the hands and feet, pain and sensitivity as well as tingling sensations. Among the numerous positive effects that carnitine (discussed below) provides is prevention of nerve disease associated with diabetes. Autonomic, motor and sensory nerve facilitation is provided by Colostrum. It has been established that bovine Colostrum contains IGF-1. An increase in growth hormone production may be potentiated by the use of IGF-1 containing Colostrum. Continued stimulation of growth hormone production may give rise to reversal of long term nerve damage and neuropathy.

The insulin mediators/precursors and glycemic control/potentiators are provided by magnesium and zinc. Conditions that produce lower levels of magnesium or deplete magnesium have been demonstrated in studies to be associated with increased insulin resistance and/or decreased insulin production. Zinc has demonstrated the ability to achieve better glycemic control and improvement in peripheral neuropathy.

Doctors have suggested that quercetin might help people with diabetes because of it's ability to reduce levels of sorbitol—a sugar that accumulates in nerve cells. Once the accumulation of sorbitol occurs, nerve cell hypoxia and ultimate nerve death may occur.

The microcirculation improvement/protection and anti-inflammatory action is provided by Butchers Broom Root, Horse Chestnut Extract, and Alpha Lipoic Acid.

The mitochondrial cell rehabilitation and lipid reduction is provided by Acetyl L-carnitine. Advanced glycation end products (AGES) are reduced with the usage of Acetyl L-carnitine. Pain associated with distal polyneuropathy responds to treatment with Acetyl L-carnitine. Apoptosis associated with mitochondrial dysfunction may contribute to the pathogenesis of diabetic sensory neuropathy. Elevated lipids inside blood vessels (atherosclerosis) can choke off blood supply to certain peripheral nerves. Without oxygen and nutrients, the nerves slowly die.

Nerve conduction velocity facilitation potential is provided by Inositol and Vitamin B-1 (thiamine.) An increase in the amplitude of evoked action potentials of the median, sural, and popliteal nerves of diabetic patients were correlated with the usage of orally administered Inositol supplementation. It is well known that Inositol and Vitamin B-1 (thiamine) affect nerve transmissions.

Pancreatic stem cell/insulin producing complement is provided by L-Taurine. Deficiency of Taurine during pregnancy could contribute to type 2 diabetes later in life. Supplementing Taurine could help stave off the late-onset form of the disease.

The preferred range of ingredients and a preferred embodiment for a capsule form of the formulation is as follows, with all percentages expressed by weight:

Nerve and Circulatory Nutritional Formulation

| Ingredient | Amount | | % By Weight | Caps Pref % |
|---|---|---|---|---|
| Vitamin A (as palmitate) | 0-5,000 | IU | 0-0.375 | 0.39 |
| Vitamin C (as ascorbic acid) | 25-400 | mg | 2.5-40 | 10 |
| Vitamin E (as d-alpha tocopherol succinate) | 25-400 | IU | 0.01-0.2 | 0.1 |
| Thiamine HCL | 1-150 | mg | 0.1-15 | 3.9 |
| Riboflavin | 1-150 | mg | 0.1-15 | 1 |
| Niacin (as inositol hexanicinate) | 1-750 | mg | 0.1-75 | 5.9 |
| Vitamin B6 (as pyridoxine HCL) | 1-150 | mg | 0.1-15 | 1.9 |
| Vitamin B12 (as methylcobalamin) | 100-5000 | mcg | 0.01-0.5 | .19 |
| Biotin (as d-biotin) | 1-5000 | mcg | 0.0001-0.5 | .19 |
| Folic Acid | 10-400 | mg | 1-40 | .08 |
| Magnesium (as magnesium oxide) | 0-750 | mg | 0-75 | 10 |
| Zinc (as zinc amino acid chelate) | 0-250 | mg | 0-25 | 1.9 |
| Copper (as citrate) | 1-4000 | mcg | 0.0001-0.4 | .05 |
| Acetyl-L-Carnitine | 1-700 | mg | 0.1-70 | 19 |
| Horse Chestnut Extract | 1-500 | mg | 0.1-50 | 19 |
| Colostrum | 1-500 | mg | 0.1-50 | 10 |
| L-Taurine | 1-400 | mg | 0.1-40 | 4.9 |
| Butcher's Broom Root | 1-500 | mg | 0.1-50 | 3.9 |
| Alpha Lipoic Acid | 1-250 | mg | 0.1-25 | 2.9 |
| Betaine HCL | 1-100 | mg | 0.1-10 | .39 |
| Quercetin | 1-100 | mg | 0.1-10 | .39 |
| Magnesium stearate (inert) | | | | 2.9 |

In this embodiment, Vitamin A, C, E, alpha lipoic acid and zinc provide antioxidant/free radical scavenging action. Stimulation of the healing process, microcirculation and anti-inflammatory activity are due to Butchers Broom Root and Horse Chestnut Extract and alpha lipoic acid. Niacin (Vitamin B-3) acts as a vasodilator. Vitamin B-12 (Methylcobalamin), Vitamin B-1 (Thiamine), Inositol, and colostrum potentiates nerve growth, conduction and regeneration. Magnesium and Zinc help to achieve glycemic control. Quercetin acts as a sorbitol inhibitor. Acetyl L-carnitine provides for lipid reduction and mitochondrial activation. L-Taurine acts as a pancreatic stem cell support element.

The invention, as portrayed in the above embodiment, is ideal for the treatment of peripheral neuropathy, vascular insufficiency, extremity numbness, burning feet, extremity hypersensitivity, pins-and-needles sensations, tingling sensations, crawling and prickling sensations, extremity pain, dizziness, and muscle weakness.

The invention could be modified to include various ingredients such as evening primrose oil, flax seed oil, borage oil, epa/dha (salmon oil) and others.

Many alterations may be made by those having ordinary skill in the art without departing from the spirit and scope of the invention. Although the present invention has been described with reference to preferred embodiments, numerous modifications and variations can be made, some of which were described above, and the results will still fall within the scope of the invention.

We claim:

1. A non-toxic combination of ingestable nutrient formulations for the use in the treatment of a wide variety of conditions associated with complications arising from diabetes and circulatory problems, comprising an effective amount of antioxidant portion, an effective amount of anti-inflammatory portion, an effective amount of circulatory enhancement portion, an effective amount of vasodilator portion, an effective amount of nerve growth, conduction and regeneration portion, an effective amount of glycemic control portion, an effective amount of sorbitol inhibitor portion, an effective amount of lipid reduction portion, an effective amount of mitochondrial activation portion, and an effective amount of pancreatic stem cell support element portion.

2. The formulation as set forth in claim 1, said antioxidant portion consisting essentially of from 0% to 20% Vitamin C, 0% to 5% Vitamin E, 0% to 5% Vitamin A, 0% to 25% alphalipoic acid and 0% to 25% zinc, determined as a percentage of total weight of the formula.

3. The formulation as set forth in claim 1, said anti-inflammatory portion consisting essentially of from 0% to 50% butchers broom root, 0% to 50% horse chestnut extract and 0% to 25% alpha lipoic acid, determined as a percentage of the total weight of the formula.

4. The formulation as set forth in claim 1, said circulatory enhancement portion consisting essentially of from 0% to 50% butchers broom root, 0% to 50% horse chestnut extract and 0% to 25% alpha lipoic acid, determined as a percentage of the total weight of the formula.

5. The formulation as set forth in claim 1, said vasodilator portion consisting essentially of from 0% to 75% niacin (Vitamin B-3), 0% to 15% thiamin (Vitamin B-1) and 0% to 50% horse chestnut extract, determined as a percentage of the total weight of the formula.

6. The formulation as set forth in claim 1, said nerve growth, conduction, and regeneration portion consisting essentially of from 0% to 90% methylcobalamin (Vitamin B-12), 0% to 50% colostrum and 0% to 15% thiamin (Vitamin B-1), determined as a percentage of the total weight of the formula.

7. The formulation as set forth in claim 1, said glycemic control portion(s) consisting essentially of from 0% to 75% magnesium and 0% to 25% zinc, determined as a percentage of the total weight of the formula.

8. The formulation as set forth in claim 1, said sorbitol inhibitor portion consisting essentially of from 0% to 10% quercetin, determined as a percentage of the total weight of the formula.

9. The formulation as set forth in claim 1, said lipid reduction portion consisting essentially of from 0% to 70% acetyl L-carnitine, determined as a percentage of the total weight of the formula.

10. The formulation as set forth in claim 1, said mitochondrial activation portion consisting essentially of from 0% to 70% acetyl L-carnitine, determined as a percentage of the total weight of the formula.

11. The formulation as set forth in claim 1, said pancreatic stem cell support portion consisting essentially of from 0% to 40% L-taurine, determined as a percentage of the total weight of the formula.

12. The formulation as set forth in claim 1 wherein the antioxidant portion is taken from the group of Vitamin D, glutathione, and pycnogenol.

13. The formulation as set forth in claim 1, wherein the anti-inflammatory portion is taken from the group of alphabisabolol and chemazulene.

14. The formulation as set forth in claim 1, wherein the circulatory enhancement portion is taken from the group of nutmeg oil and cinnamon oil.

15. The formulation as set forth in claim 1, wherein the vasodilator portion is cinnamaldehyde.

16. The formulation as set forth in claim 1, wherein the nerve conduction, growth and regeneration potion is taken from the group of Acetyl-L-Carnitine, N-Acetyl Cysteine, Gamma-Linoleic Acid (GLA), Evening Primrose Oil and SAMe.

17. The formulation as set forth in claim 1, wherein the glycemic control portion is taken from the group of fenugreek and chromium.

18. The formulation as set forth in claim 1, wherein the sorbitol inhibitor portion is Acetyl-L-Carnitine.

19. The formulation as set forth in claim 1, wherein the lipid reduction portion is taken from the group of Chitosan, Choline, Biotin, PAPA, Lecithin, Inositol and Phosphatydilcholine.

20. The formulation as set forth in claim 1, wherein the mitochondrial activation portion is taken from the group of Glutathione, N-Acetyl Cysteine, Carnosine, SAMe, Pyridoxyl-5-Phosphate and Coenzyme Q10.

21. An ingestible nutrient formulation comprising by weight about 0.39% Vitamin A, about 10% Vitamin C, about 0.1% Vitamin E, about 3.9% Thiamine HCL, about 1% Riboflavin, about 5.9% Niacin, about 1.9% Vitamin B6, about 0.19% Vitamin B12, about 0.19% Biotin, about 0.08% Folic Acid, about 10% Magnesium, about 1.9% Zinc, about 0.05% Copper, about 19% Acetyl-L-Carnitine, about 19% Horse Chestnut Extract, about 10% Colostrum, about 4.9% L-Taurine, about 3.9% Butcher's Broom Root, about 2.9% Alpha Lipoic Acid, about 0.39% Alpha Lipoic Acid, about 1% Betaine HCL, about 0.39% Quercetin and the remainder an inert ingredient.

22. The formulation of claim 21 wherein the inert ingredient is Magnesium Stearate.
